(19) United States
(12) Patent Application Publication
(10) Pub. No.: US 2009/0269282 A1
(43) Pub. Date: Oct. 29, 2009

(54) Methods for treating, preventing and diagnosing procine ttv infection

(76) Inventor: John A. Ellis, Saskatoon (CA)

Correspondence Address: ROBINS & PASTERNAK, 1731 EMBARCADERO ROAD, SUITE 230, PALO ALTO, CA, 94303, US

(21) Appl. No.: 12/311,260

(22) PCT Filed: Sep. 25, 2007
(86) PCT No.: PCT/US2007/020793

§ 371 Date: Jul. 2, 2009

Related U.S. Application Data
(60) Provisional application No. 60/936,193, filed on Jun. 19, 2007.
(60) Provisional application No. 60/849,731, filed on Oct. 5, 2006.

(51) Int. Cl. A61K 49/00; A61M 37/00; A61K 39/12; G01N 33/53; G01N 33/569; C12Q 1/70

(52) U.S. Cl. 424/9.2; 604/522; 424/204.1; 424/186.1; 435/7.1; 435/7.9; 435/5

(57) ABSTRACT

Compositions and methods for treating, preventing and diagnosing Torque teno virus (TTV) infection in pigs are disclosed. Also described are methods of identifying compounds for the treatment and prevention of TTV infection, as well as TTV animal models and methods of producing the same.

### CROSS-REFERENCE TO RELATED APPLICATIONS

This application claims the benefit pursuant to 35 U.S.C. §119(e)(1) of U.S. application Ser. Nos. 60/849,731, filed Oct. 5, 2006, and 60/936,193, filed Jun. 19, 2007, which applications are incorporated herein by reference in their entireties.

### TECHNICAL FIELD

The present invention relates generally to viral pathogens. In particular, the invention pertains to porcine Torque teno virus (TTV), methods of treating, preventing and diagnosing TTV infection and TTV-related disorders in pigs, and to animal models for use in studying TTV and TTV-related disorders.

### BACKGROUND

Torque teno virus (TTV), also known as transfusion-transmitted virus, belongs to the Anellovirus floating genus and has been provisionally assigned to the Circoviridae family. TTV was originally isolated from the blood of a human patient with a post-transfusion hepatitis of unknown etiology. Nishizawa et al., Biochem. Biophys. Res. Commun. (1997) 241:92-97. TTV has now been identified in a number of animals in addition to humans, including in pigs. Porcine TTV has been isolated in several countries and sequence analyses have shown that the various strains share between 71% to 100% nucleotide sequence identity. McKeown et al., Vet. Microbiol. (2004) 104:113-117.

TTV is a small, non-enveloped virus with a single-stranded circular DNA genome of negative polarity. The genome includes an untranslated region and at least three major overlapping open reading frames. Biagini, P., Vet. Micorbiol. (2004) 98:95-101. ORF1 encodes a DNA replicase, ORF2 a nucleocapsid protein and ORF3 a protein with apoptotic activity.

TTV infections are common amongst asymptomatic humans but the incidence of TTV viremia tends to be elevated in patients with a variety of different clinically evident diseases, including viral hepatitis, HIV/AIDs, asthma and related childhood respiratory conditions and renal disease. However, attempts to link TTV infection with human disease are confounded by the high incidence of asymptomatic TTV viremia in control cohort population(s), the remarkable genomic diversity within the TTV viral family, the inability to propagate the agent in vitro and the lack of animal model(s) of TTV disease (Yzebe et al., Panminerva Med. (2002) 44:167-177; Biagini, P., Vet. Microbiol. (2004) 98:95-101). While TTV appears to be acquired by oronasal or fecal-oral infection, mother-to-infant and/or in utero transmission have also been documented (Gerner et al., Ped. Infect. Dis. J. (2000) 19:1074-1077). Infected persons are characterized by a prolonged (months to years) TTV viremia. Humans may be co-infected with more than one TTV genogroup (Saback, et al., Scad. J. Infect. Dis. (2001) 33:121-125). There is a suggestion that these genogroups can recombine within infected humans (Rey et al., Infect. (2003) 31:226-233). Although the data are fragmentary, there is also evidence that an antibody response is made to TTV viral nucleocapsid protein during the course of the infection and that circulating TTV-antibody immune complexes may develop in infected individuals (Yzebe et al., Panminerva Med. (2002) 44:167-177; Biagini, P., Vet. Microbiol. (2004) 98:95-101). While levels of viral DNAs approaching 10¹⁰ copies per ml have been reported (Rey et al., Infect. (2003) 31:226-233), most investigators use the more sensitive nested (n) polymerase chain reaction (PCR) to detect TTV DNAs in sera and tissue extracts. Target sequences vary, but primer pairs specific for the untranslated region (UTR) of the DNA genome are the most broadly reactive and are used for screening suspect samples that may contain multiple TTV genogroups (Rey et al., Infect. (2003) 31:226-233; Leary et al., J. Gen. Virol. (1999) 80:2115-2120). Because the double stranded isoform (replicative) intermediates have been found in liver, peripheral blood mononuclear cells and bone marrow, these are the presumed sites of viral replication in vivo (Kikuchi et al., J. Med. Virol. (2000) 61:165-170; Okamoto et al., Biochem. Biophys. Res. Commun. (2002) 270:657-662; Rodriguez-lnigo et al., Am. J. Pathol. (2000) 156:1227-1234). Since no disease processes or syndromes have been directly linked to TTV most investigators consider the TTVs to be “orphan” or avirulent viruses of humans and animals, that is, a virus still waiting to be clearly linked to a given disease (Biagini, P., Vet. Microbiol. (2004) 98:95-101; Irshad et al., World J. Gastroenterol. (2006) 12:5122-5134; Simmonds et al., J. Infect. Dis. (1999) 180:1748-1750; Zein, N., J. Pediat. (2000) 82:379-383; Beninelli et al., Clin. Microbiol. Rev. (2001) 14:98-113).

Porcine TTV is ubiquitous and PCR-detection of the virus in serum samples collected from various geographical regions shows prevalence in pigs ranging from 33 to 100%. McKeown et al., Vet. Microbiol. (2004) 104:113-117. Two genogroups (1 and 2) have been identified in pigs (Neil et al., J. Gen. Virol. (2005) 86:1343-1347); roughly one-half of swine sera examined contained both TTV genogroups (Kekarainen et al., J. Gen. Virol. (2006) 87:833-837). A higher prevalence of TTV infection in the sera from post-weaning multisystemic wasting syndrome (PMWS) pigs has been reported (Kekarainen et al., J. Gen. Virol. (2006) 87:833-837). However, a correlation between PMWS clinical symptoms and TTV has not been shown. Moreover, there is a higher occurrence of other viruses in PMWS-affected pigs that do not contribute to the PMWS disease state. Segales and Domingo, Vet. Q. (2002) 24:109-124. Thus, to date, there is no clear relationship between porcine TTV and any particular pathology. Moreover the role of TTV during co-infection with other pathogens remains unknown.

Porcine circovirus-associated disease (PCVAD), also termed porcine circovirus disease (PCVD) refers to a complex of swine diseases that includes porcine circovirus type 2 (PCV2) and its primary disease manifestation postweaning multisystemic wasting syndrome (PMWS), enteritis, respiratory disease in growing and fattening pigs associated with PCV2 in lung lesions, and porcine dermatitis and nephropathy syndrome (PDNS). PCVAD appears to require multiple components to develop. For example, PCV-2 is a factor, but rarely if ever by itself is it sufficient to cause clinical signs of disease. Other infections appear to be necessary, such as porcine reproductive and respiratory syndrome virus (PRRSV), swine influenza virus and Mycoplasmal pneumonia. Environmental stressors also can be factors in PCVAD.

The common underlying feature of all of these co-factors is upregulation of immune responses during covalescence, which is manifest as reactive systemic lymphoid hypertrophy and hyperplasia. Intentional immunizations with immunogens emulsified in a strong adjuvant (incomplete Freund's adjuvant) mimic this lympho-proliferative reaction and this has been used to induce PMWS in PCV2-infected gnotobiotic piglets, absent other swine infectious disease agents (Krakowka et al., Vet. Pathol. (2001) 38:31-42). This effect is also seen in piglets immunized with certain Mycoplasma hyponeumoniae bacterins under field (Kyriakis et al., J. Comp. Pathol. (2002) 126:3846) and experimental (Krakowka et al., Can. Vet. J. (2007) 48:716-724) conditions.

However, many epidemics of PMWS have not been associated with known swine infectious diseases, changes in management or vaccinations, an observation that suggests that additional unidentified factors are operable in the disease complex.

These associated diseases are rapidly becoming a major threat to the health of swine in the United States and other countries. New, harsher episodes of PCVAD are surfacing throughout the world, including in Canada, the U.S., the Far East, New Zealand and Europe, seriously affecting the supply of pigs. Mortality rates range from 18-50% and can run as high as 70% and morbidity rates are approximately 4-30%, with episodes occurring beyond 50%. Miller, M., Pork Magazine, “PCVAD:What's Known and Unknown” (Jul. 1, 2006).

Due to the tremendous economic impact of PCVAD, methods of treating, preventing and diagnosing infection are needed.

### SUMMARY OF THE INVENTION

The inventors herein have surprisingly linked TTV to PMWS and have discovered that TTV is another viral infection that potentiates PCV2-induced PMWS. This discovery evidences that the TTVs contribute to the clinical spectrum of disease associated with other single agent infections in swine and is therefore useful in the development of vaccines against PCVAD. The potentiating effects of g1-TTV infection upon manifestations of PCV2-induced PMWS are shown below. Moreover, it has been found that the sequence of infection in dually infected piglets determines clinical expression of disease.

The inventors have also produced animal models of TTV infection. In particular, the presence of swine genogroup 1 TTV (g1-TTV) DNA by nPCR in the sera of PCV2-negative healthy pigs is demonstrated, as well as infectivity of g1-TTV by serial passage of TTV-positive plasmas through gnotobiotic swine. A reproducible spectrum of lesions was observed in TTV-infected gnotobiotes that were not present in uninfected gnotobiotic control piglets. That TTV replicated in pigs was evident when serum TTV DNAs were readily detected by conventional PCR. The inventors herein have therefore shown for the first time in any species that TTV is pathogenic in the host species of origin if host conditions are appropriately manipulated. Moreover, as described further below, gnotobiotic swine and porcine TTV infection provide an animal model of porcine and human TTV infection. Gnotobiotics represent a new approach to the study of the TTVs. By using gnotobiotic animals, environmental influences can be excluded, including inadvertent transmission of TTVs contained in the environment to the experimental subjects. Gross and histologic changes observed in pathogen-infected gnotobiotes are direct reflections of the interactions between the agent and the host and, by definition, are not confounded by external variables such as commensal and concurrent infections, dietary differences and environmental pressures.

Thus, the present invention relates to the use of TTV preparations in the treatment, prevention and/or diagnosis of TTV-related diseases, such as in the treatment, prevention and/or diagnosis of PCVAD-associated diseases, including PMWS, PCV2, PDNS, reproductive disorders, enteritis, and respiratory disease in growing and fattening pigs. Attenuated, inactivated or subunit vaccines, including immunogens and mixtures of immunogens derived from porcine TTV isolates are used to provide protection against subsequent infection with TTV and are therefore useful for protection against diseases related to TTV infection. The present invention thus provides a commercially useful method of treating, preventing and/or diagnosing TTV infection in swine.

Accordingly, in one embodiment, the subject invention is directed to a composition comprising a pharmaceutically acceptable vehicle and at least one porcine TTV immunogen selected from the group consisting of an inactivated immunogenic porcine TTV, an attenuated immunogenic porcine TTV and an isolated immunogenic porcine TTV polypeptide. In certain embodiments, the composition further comprises an adjuvant.

In additional embodiments, the invention is directed to a method of treating or preventing TTV infection in a porcine subject comprising administering to the subject a therapeutically effective amount of a composition as described above.

In further embodiments, the invention is directed to a method of treating or preventing a PCVAD in a porcine subject comprising administering to the subject a therapeutically effective amount of a composition as described above. In certain embodiments, the PCVAD is postweaning multisystemic wasting syndrome (PMWS) and/or porcine dermatitis and nephropathy syndrome (PDNS).

In yet further embodiments, the invention is directed to a method of producing a composition comprising (a) providing at least one porcine TTV immunogen selected from the group consisting of an inactivated immunogenic porcine TTV, an attenuated immunogenic porcine TTV and an isolated immunogenic porcine TTV polypeptide; and
(b) combining the TTV immunogen with a pharmaceutically acceptable vehicle. In certain embodiments, the method further comprises providing an adjuvant.

In certain embodiments, the compositions used in the methods include additional immunogens from pathogens that cause disease in pigs, such as but not limited to, immunogens from porcine parvovirus, porcine circovirus, porcine reproductive and respiratory syndrome virus, swine influenza, pseudorabies virus, pestivirus which causes porcine swine fever, porcine lymphotropic herpesviruses (PLHV1 and PLHV2), Mycoplasma spp, Helicobacter spp, Campylobacter spp, Lawsonia spp, Actinobacillus pleuropneumoniae, Haemophilus parasuis, Streptococcus spp, Pasteurella spp, Salmonella spp, E. coli, Clostridium spp, Eryspelothrix rhusiopathiae.

In additional embodiments, the invention is directed to a method of detecting TTV infection in a vertebrate subject comprising (a) providing a biological sample from the subject; and (b) reacting the biological sample with at least one isolated immunogenic porcine TTV polypeptide, under conditions which allow TTV antibodies, when present in the biological sample, to bind with the TTV polypeptide, thereby detecting the presence or absence of TTV infection in the subject. In certain embodiments, the method further comprises (c) removing unbound antibodies; (d) providing one or more moieties capable of associating with said bound antibodies; and (e) detecting the presence or absence of the one or more moieties, thereby detecting the presence or absence of TTV infection.

In certain embodiments, the detectable label is a fluorescer or an enzyme. In additional embodiments, the biological sample is a porcine serum sample.

In yet an additional embodiment, the invention is directed to a method for infecting a young TTV-negative piglet, a barrier-raised specific pathogen-free piglet, or a caesarian-delivered piglet, with a porcine isolate of TTV. The method comprises (a) isolating TTV from a porcine subject; and (b) administering a dose of the TTV isolate to the piglet in an amount sufficient to cause TTV infection.

In further embodiments, the invention is directed to a method for evaluating the ability of a vaccine to prevent TTV infection comprising (a) administering to a porcine subject a candidate vaccine; (b) exposing the porcine subject from step (a) to a porcine TTV isolate in an amount sufficient to cause infection in an unvaccinated subject; and (c) observing the incidence of TTV infection in the porcine subject, thereby evaluating the ability of the candidate vaccine to prevent TTV infection. In certain embodiments, the porcine subject is a young TTV-negative piglet, a barrier-raised specific pathogen-free piglet, or a caesarian-delivered piglet.

In another embodiment, the invention is directed to a method for evaluating the ability of a vaccine to prevent a PCVAD comprising: (a) administering to a porcine subject a candidate vaccine; (b) exposing the porcine subject from step (a) to a porcine TTV isolate in an amount sufficient to cause infection in an unvaccinated subject; and
(c) observing the incidence of a PCVAD in the porcine subject, thereby evaluating the ability of the candidate vaccine to prevent PCVAD.

In certain embodiments, the porcine subject is a young TTV-negative piglet, a barrier-raised specific pathogen-free piglet, or a caesarian-delivered piglet. In additional embodiments, the PCVAD is PMWS, PCV2 and/or PDNS.

In another embodiment, the invention is directed to a method of identifying a compound capable of treating a porcine TTV infection. The method comprises (a) exposing a young TTV-negative piglet, a barrier-raised specific pathogen-free piglet, or a caesarian-delivered piglet, to a porcine TTV isolate in an amount sufficient to cause infection in the piglet; (b) delivering a compound or series of compounds to the infected piglet; and (c) examining the piglet from step (b) for the presence or loss of TTV and/or the development, inhibition, or amelioration of PCVAD symptoms relative to an untreated TTV-infected piglet.

These and other embodiments of the subject invention will readily occur to those of skill in the art in view of the disclosure herein.

### DETAILED DESCRIPTION OF THE INVENTION

The practice of the present invention will employ, unless otherwise indicated, conventional techniques of molecular biology, microbiology, bacteriology, virology, recombinant DNA technology, and immunology, which are within the skill of the art. Such techniques are explained fully in the literature. See, e.g., Sambrook, Fritsch & Maniatis, Molecular Cloning: A Laboratory Manual, current edition; Fundamental Virology, current edition, vol. I & II (B. N. Fields and D. M. Knipe, eds.); DNA Cloning, Vols. I and II (D. N. Glover ed. current edition); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

All publications, patents and patent applications cited herein, whether supra or infra, are hereby incorporated by reference in their entirety.

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a TTV immunogen” includes a mixture of two or more such immunogens, and the like.

By “TTV infection” is meant any disorder caused directly or indirectly by a Torque teno virus, including without limitation, infection caused by any of the known TTV strains and isolates included in any of the TTV genogroups. Currently, the virus is subdivided into approximately 40 genotypes which cluster in five clearly distinct phylogenetic groups, designated 1 to 5 (Biagini et al., “Anellovirus,” p. 335-341 in Fauquet et al. eds. Virus taxonomy, 8th report of the International Committee for the Taxonomy of Viruses. Elsevier/Academic Press, New York (2004); Devalle and Niel, J. Med. Virol. (2004) 72:166-173; Hino, S., Rev. Med. Virol. (2002) 12:151-158; Nishizawa et al., Biochem. Biophys. Res. Commun. (1997) 241:92-97; Okamoto and Mayumi, J. Gastroenterol. (2001) 36:519-529; Peng et al., Arch. Virol. (2002) 147:21-41). Particular porcine isolates include but are not limited to Sd-TTV31, Sd-TTV1p, Sd-TTV2p, TTV isolates 3h and 2h (see, e.g., Niel et al., J. Gen. Virol. (2005) 86:1343-1347; Okamoto et al., J. Gen. Virol. (2002) 83:1291-1297). Such disorders include, without limitation, Porcine circovirus associated disease (PCVAD) such as porcine circovirus type 2 (PCV-2), postweaning multisystemic wasting syndrome (PMWS), enteritis, respiratory disease in growing and fattening pigs, reproductive disorders, and porcine dermatitis and nephropathy syndrome (PDNS).

The term also intends subclinical disease, e.g., where TTV infection is present but clinical symptoms of disease have not yet manifested themselves. Subjects with subclinical disease can be asymptomatic but may nonetheless be at risk of developing any of the above disorders.

By “young piglet” is meant a piglet from birth to six weeks of age, preferably from birth to three weeks of age.

The term “polypeptide” when used with reference to a TTV immunogen, refers to the immunogen, whether native, recombinant or synthetic, which is derived from any TTV strain. The polypeptide need not include the full-length amino acid sequence of the reference molecule but can include only so much of the molecule as necessary in order for the polypeptide to retain immunogenicity and/or the ability to treat, prevent or diagnose TTV infection, as described below. Thus, only one or few epitopes of the reference molecule need be present. Furthermore, the polypeptide may comprise a fusion protein between the full-length reference molecule or a fragment of the reference molecule, and another protein that does not disrupt the reactivity of the TTV polypeptide. It is readily apparent that the polypeptide may therefore comprise the full-length sequence, fragments, truncated and partial sequences, as well as analogs and precursor forms of the reference molecule. The term also intends deletions, additions and substitutions to the reference sequence, so long as the polypeptide retains immunogenicity.

Thus, the full-length proteins and fragments thereof, as well as proteins with modifications, such as deletions, additions and substitutions (either conservative or non-conservative in nature), to the native sequence, are intended for use herein, so long as the protein maintains the desired activity. These modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the biological activity, are contemplated for use herein.

The term “analog” refers to biologically active derivatives of the reference molecule, or fragments of such derivatives, that retain activity, as described above. In general, the term “analog” refers to compounds having a native polypeptide sequence and structure with one or more amino acid additions, substitutions and/or deletions, relative to the native molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

A “purified” protein or polypeptide is a protein which is recombinantly or synthetically produced, or isolated from its natural host, such that the amount of protein present in a composition is substantially higher than that present in a crude preparation. In general, a purified protein will be at least about 50% homogeneous and more preferably at least about 80% to 90% homogeneous.

By “biologically active” is meant a TTV protein that elicits an immunological response, as defined below.

By “subunit vaccine composition” is meant a composition containing at least one immunogen, but not all immunogens, derived from or homologous to an immunogen from TTV. Such a composition is substantially free of intact virus. Thus, a “subunit vaccine composition” is prepared from at least partially purified (preferably substantially purified) immunogens from TTV, or recombinant analogs thereof. A subunit vaccine composition can comprise the subunit antigen or antigens of interest substantially free of other antigens or polypeptides from the pathogen. Representative immunogens include those derived from any of ORFs 1, 2 or 3 of the TTV genome, such as immunogens from ORF2, the nucleocapsid protein, including the full-length protein or fragments thereof. Moreover, immunogens from multiple genogroups or isolates can be present. For example, the proteins expressed from ORF2 of porcine genogroups 1 and 2 (Neil et al., J. Gen. Virol. (2005) 86:1343-1347) may both be present in the subunit composition, as well as additional immunogens from TTV or other viruses. Also encompassed is the use of consensus sequences from any of the above ORFs based on multiple genotypes of TTV, such as but not limited to TTV genotypes 1 and 2.

By “epitope” is meant a site on an antigen to which specific B cells and T cells respond. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-10 such amino acids. Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:39984002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or gamma delta (γδ) T cells, directed specifically to an antigen or antigens included in the composition or vaccine of interest. Preferably, the host will display a protective immunological response to the TTV immunogen(s) in question, e.g., the host will be protected from subsequent infection by the pathogen and such protection will be demonstrated by either a reduction or lack of symptoms normally displayed by an infected host or a quicker recovery time.

The terms “immunogenic” protein or polypeptide refer to an amino acid sequence which elicits an immunological response as described above. An “immunogenic” protein or polypeptide, as used herein, includes the full-length sequence of the particular TTV immunogen in question, including any precursor and mature forms, analogs thereof, or immunogenic fragments thereof. By “immunogenic fragment” is meant a fragment of the TTV immunogen in question which includes one or more epitopes and thus elicits the immunological response described above.

Immunogenic fragments, for purposes of the present invention, will usually be at least about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the TTV immunogen in question.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by Intelligenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art.

Alternatively, homology can be determined by hybridization of polynucleotides under conditions which form stable duplexes between homologous regions, followed by digestion with single-stranded-specific nuclease(s), and size determinati on of the digested fragments. DNA sequences that are substantially homologous can be identified in a Southern hybridization experiment under, for. example, stringent conditions, as defined for that particular system. Defining appropriate hybridization conditions is within the skill of the art. See, e.g., Sambrook et al., supra; DNA Cloning, supra; Nucleic Acid Hybridization, supra.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors.

By “recombinant vector” is meant a vector that includes a heterologous nucleic acid sequence which is capable of expression in vitro or in vivo.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52 :456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties into suitable host cells.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-24hiouraci l, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “3 prime (3′)” or “5 prime (5′)” relative to another sequence, it is to be understood that it is the position of the sequences in the “sense” or “coding” strand of a DNA molecule that is being referred to as is conventional in the art.

The terms “effective amount” or “therapeutically effective amount” of a composition or agent, as provided herein, refer to a nontoxic but sufficient amount of the composition or agent to provide the desired “therapeutic effect,” such as to elicit an immune response as described above, preferably preventing, reducing or reversing symptoms associated with the TTV infection. This effect can be to alter a component of a disease (or disorder) toward a desired outcome or endpoint, such that a subject's disease or disorder shows improvement, often reflected by the amelioration of a sign or symptom relating to the disease or disorder. For example, a representative therapeutic effect can render the subject negative for TTV infection when samples from pigs are cultured for TTV. Similarly, biopsies indicating lowered IgG, IgM and IgA antibody production directed against TTV can be an indication of a therapeutic effect. Similarly, decreased serum antibodies against TTV are indicative of a therapeutic effect. Reduced symptoms of PCVAD-related disease are also indicative of a therapeutic effect. The exact amount required will vary from subject to subject, depending on the species, age, and general condition of the subject, the severity of the condition being treated, and the particular components of the composition administered, mode of administration, and the like. An appropriate “effective” amount in any individual case may be determined by one of ordinary skill in the art using routine experimentation.

“Treatment” or “treating” TTV infection includes: (1) preventing the TTV disease, or TTV-associated disease that includes another pathogen, such as PCV2, or (2) causing disorders related to TTV infection to develop or to occur at lower rates in a subject that may be exposed to TTV, (3) reducing the amount of TTV present in a subject, and/or reducing the symptoms associated with TTV infection.

As used herein, a “biological sample” refers to a sample of tissue or fluid isolated from an individual, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

As used herein, the terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof which is capable of exhibiting fluorescence in the detectable range. Particular examples of labels which may be used under the invention include fluorescein, rhodamine, dansyl, umbelliferone, Texas red, luminol, acradimum esters, NADPH and α-β-galactosidase.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Central to the present invention is the use of TTV and immunogens derived therefrom, to develop immunogenic compositions for use as vaccines and diagnostics to prevent, treat and diagnose TTV infection. As explained above, TTV is associated with PCVAD-associated diseases. Thus, the invention provides compositions and methods for preventing, treating and diagnosing such diseases as PMWS and PDNS.

New animal models useful for studying the pathogenesis, treatment and prevention of TTV infection in pigs can be developed. For example, young TTV-negative piglets, barrier-raised specific pathogen-free piglets, or caesarian-delivered piglets can be used to study the ability of various TTV vaccines to prevent TTV infection. Additionally, young TTV-negative piglets, barrier-raised specific pathogen-free piglets, or caesarian-delivered piglets infected with TTV can be used to screen various compounds for their ability to treat TTV infection.

In order to further an understanding of the invention, a more detailed discussion is provided below regarding animal models, TTV immunogens, as well as various uses thereof.

TTV Animal Models

Swine are monogastric omnivores with gastric anatomy and physiology that closely replicates humans. Young TTV-negative piglets, barrier-raised specific pathogen-free piglets, or caesarian-delivered piglets may be suited for studying TTV infection and therefore for identifying vaccine candidates, such as vaccines including one or more immunogens derived from TTV, useful for preventing TTV infection in pigs. Barrier-born pigs are free from the specific pathogens affecting individual herds. Caesarian and barrier derived animals have been shown to have a markedly reduced prevalence of PCVs and PLHVs. See, e.g., Tucker et al., Xenotransplantation (2003) 10: 343-348.

Thus, a preferred use for the animal models of the invention is the development of vaccines for use in the prevention and/or treatment of TTV infection in pigs and diseases associated therewith.

In this context, young TTV-negative piglets, barrier-raised specific pathogen-free piglets, or caesarian-delivered piglets are administered the vaccine candidate at least once, and preferably boosted with at least one additional immunization. For example, piglets can be administered a vaccine composition to be tested at 1-5 days of age, followed by a subsequent boost 5-10 days later, and optionally a third immunization 5-10 days following the second administration. Piglets can be vaccinated as many times as necessary. The vaccinated piglets are then exposed to TTV approximately 3-20 days later, such as 4-10 days following the last immunization. Typically, vaccinated piglets are parenterally or orally administered from 10² to 10⁸ pfu, more particularly from 10⁵ to 10⁷ pfu of TTV, and indicia of TTV infection are monitored.

Such indicia include TTV viral titer, as well as symptoms of PCVAD disease, such as symptoms of PMWS, respiratory and enteric problems, wasting, lymphoid lesions, granulomatous inflammation, the presence of PCV2 antigen, and histopathology and PCV2 detection by in situ hybridization. See, e.g., Segales et al., Swine Health Prod. (2002) 10:277-281 and Rosell et al., J. Comp. Pathol. (1999) 120:59-78. Similarly, signs of PDNS can be monitored. The most obvious signs are red-purple blotches on the skin of pigs that are often slightly raised. Such blotches tend to be most obvious on the hind legs, loin, scrotum and ears, but can extend over the abdomen, flanks and fore legs eventually covering the whole body. The lesions become crusty and brown after a few days. Pigs are lethargic and elevated temperatures may also be present. In acute cases, pigs have swollen legs leading to lameness. Respiratory distress and/or scouring may also be observed.

Alternatively, young TTV-negative piglets, barrier-raised specific pathogen-free piglets, or caesarian-delivered piglets can first be infected with TTV in order to establish TTV infection. For example, piglets can be parenterally inoculated at 1-5 days of age with TTV, in an amount sufficient to cause infection, such as with 10⁴ to 10¹⁰, or more pfu more particularly 10⁴ to 10¹⁰, pfu of TTV. The presence of TTV infection can be confirmed by examining biological samples, such as serum samples, for virus, e.g., by using PCR techniques well known in the art, and/or for signs of infection as described above.

Once the viral infection has been established, a compound or a series of compounds can be delivered to the infected piglet at various times and in various dosages, depending on the particular goals of the screen. In a variation of this procedure, it may be desirable to administer TTV with a compound to determine whether, relative to control animals, the compound can effectively prevent in vivo the initial viral infection and/or the subsequent establishment of infection or pathogenesis.

Thus, the infected piglets can be used to screen for compounds and conditions which prevent TTV infection, such as compounds and conditions that block entry of TTV into host cells and/or that ameliorate the TTV-associated pathogenesis seen with PCVAD-associated diseases. The efficacy of the compound or compounds can be assessed by examining at selected times biological samples from the infected animals for the presence or loss of TTV and/or the development, inhibition, or amelioration of PCVAD-associated pathologies relative to appropriate control animals, for example, untreated TTV-infected animals. The animal models described herein therefore provide the ability to readily assess the efficacy of various drugs or compounds based on different modes of administration and compound formation.

In addition to using the TTV-infected animals to screen for therapeutic compounds, these animals can also be used to screen for conditions or stimuli which effect a block in or ameliorate TTV infection and/or associated diseases. Such stimuli or conditions include environmental or dietary changes, or combinations of various stimuli or conditions which result in stress on the animal. Thus, for example, TTV-infected animals can be exposed to a selected stimulus or condition, or a combination of stimuli or conditions, to be tested. Biological samples of exposed animals are then examined periodically for a change in the number of TTV and/or the associated disease state relative to non-exposed control animals.

TTV Compositions

The animal models described herein can be used to identify TTV immunogenic compositions, useful for diagnosing, treating and/or preventing TTV infection in swine. As explained above, TTV compositions for use as vaccines and diagnostics can include attenuated or inactivated virus. Alternatively, subunit compositions, including isolated TTV immunogens, such as immunogens derived from any of the ORFs, particularly the nucleocapsid protein, encoded by ORF2 of the virus, can also be provided. Immunogens from ORF1 and ORF3 may also find use herein. Proteins including consensus sequences derived from multiple genogroups, such as porcine genogroups 1 and 2, can also be used.

TTV compositions may be derived from any TTV strain and isolate in any of the TTV genogroups. A number of TTVs are known and described in, e.g., Biagini et al., “Anellovirus,” p. 335-341 in Fauquet et al. eds. Virus taxonomy, 8th report of the International Committee for the Taxonomy of Viruses. Elsevier/Acadmeic Press, New York (2004); Devalle and Niel, J. Med. Virol. (2004) 72:166-173; Hino, S., Rev. Med. Virol. (2002) 12:151-158; Nishizawa et al., Biochem. Biophys. Res. Commun. (1997) 241:92-97; Okamoto and Mayumi, J. Gastroenterol. (2001) 36:519-529; and Peng et al., Arch. Virol. (2002) 147:21-41.

Particular porcine isolates include but are not limited to Sd-TTV31, Sd-TTV1p, Sd-TTV2p, TTV isolates 3 h and 2 h (see, e.g., Niel et al., J. Gen. Virol. (2005) 86:1343-1347; Okamoto et al., J. Gen Virol. (2002) 83:1291-1297). The genomic sequences of these isolates, including the sequences of ORF1, ORF2 and ORF3, encoding the DNA replicase, nucleocapsid protein and an apoptotic sequence, respectively, are described in, for example, Niel et al., J. Gen. Virol. (2005) 86:1343-1347; Okamoto et al., J. Gen. Virol. (2002) 83:1291-1297, as well as in NCBI Accession nos. AB076001, AY823991, AY823990, AY823989 and AY823988. Accession nos. DQ229865 and DQ229860 provide the sequences of porcine genogroup 1 TTV and porcine genogroup 2 TTV, respectively.

TTV immunogens, including whole TTV virus, can be produced using a variety of techniques. For example, the immunogens can be obtained directly from TTV that has been isolated from TTV-infected subjects, such as swine, using techniques well known in the art. Generally, TTV DNA is obtained using polymerase chain reaction (PCR) techniques, including TaqMan™ methods, using primers derived from the TTV genomic sequence, as described in Desai et al., J. Med. Virol. (2005) 77:136-143; Haramoto et al., Water Res. (2005) 39:2008-2013; Kekarainen et al., J. Gen. Virol. (2006) 87:833-837; and Martelli et al., J. Vet. Med. (2006) 53:234-238.

TTV so obtained can be replicated in various cell lines, such as hepatocyte and leukocyte cell lines, including the Chang Liver cell line, phytohemagilutinin (PHA)-stimulated peripheral blood mononuclear cell (PBMC) cultures and B lymphoblast cell lines, such as Raji, L23, L35, LCL 13271 cell lines. See, e.g., Desai et al., J. Med. Virol. (2005) 77:136-143; Bonenfant et al., Xenotransplantation (2003) 10:107-119; and Sinkora et al., Vet. Immunol. Immunopath. (2001) 80:79-91). Culture conditions for the above cell types are described in a variety of publications. The cell culture conditions to be used for the desired application (temperature, cell density, pH value, etc.) are variable over a very wide range depending on the cell line employed and can readily be adapted to the requirements of the TTV virus in question. Methods for propagating TTV in cultured cells include the steps of inoculating the cultured cells with TTV, cultivating the infected cells for a desired time period for virus propagation, such as for example as determined by virus titer or virus antigen expression (e.g., between 24 and 168 hours after inoculation) and collecting the propagated virus. The cultured cells are inoculated with the desired virus (measured by PFU or TCID50) to cell ratio of 1:500 to 1:1, preferably 1:100 to 1:5, more preferably 1:50 to 1:10. The TTV is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g., monolayers) may be removed either by freeze-thawing or by enzymatic action to increase the viral content of the harvested culture supernatants. The harvested fluids are then either inactivated or stored frozen.

Methods of inactivating or killing viruses are known in the art. Such methods destroy the ability of the viruses to infect mammalian cells. Inactivation can be achieved using either chemical or physical means. Chemical means for inactivating TTV include treatment of the virus with an effective amount of one or more of the . following agents: detergents, formaldehyde, formalin, β-propiolactone, or UV light. Other methods of viral inactivation are known in the art, such as for example binary ethylamine, acetyl ethyleneimine, or gamma irradiation.

For example, β-propiolactone may be used at concentrations such as 0.01 to 0.5%, preferably at 0.5% to 0.2%, and still more preferably at 0.025 to 0.1%. The inactivating agent is added to virus-containing cultures (virus material) prior to or after harvesting. The cultures can be used directly or cells disrupted to release cell-associated virus prior to harvesting. Further, the inactivating agent may be added after cultures have been stored frozen and thawed, or after one or more steps of purification to remove cell contaminants. β-propiolactone is added to the virus material, with the adverse shift in pH to acidity being controlled with sodium hydroxide (e.g., 1 N NaOH) or sodium bicarbonate solution. The combined inactivating agent-virus materials are incubated at temperatures from 4° C. to 37° C., for incubation times of preferably 24 to 72 hours.

Alternatively, binary ethyleneimine (BEI) can be used to inactivate virus. One representative method of inactivating TTV is as follows. BEI is made by mixing equal volumes of a 0.2 molar bromoethylamine hydrobromide solution with a 0.4 molar sodium hydroxide solution. The mixture is incubated at about 37° C. for 60 minutes. The resulting cyclized inactivant, BEI, is added to the virus materials at 0.5 to 4 percent, and preferably at I to 3 percent, volume to volume. The inactivating virus materials are held from about 4° C. to 37° C. for 24 to 72 hours with periodic agitation. At the end of this incubation, 20 ml of a sterile 1 molar sodium thiosulfate solution is added to insure neutralization of the BEI. Diluted and undiluted samples of the inactivated virus materials are added to susceptible cell (tissue) culture to detect any non-inactivated virus. The cultured cells are passaged multiple times and examined for the presence of TTV based on any of a variety of methods, such as, for example, cytopathic effect (CPE) and antigen detection. Such tests allow determination of complete virus inactivation.

Methods of purification of inactivated virus are known in the art and may include one or more of gradient centrifugation, ultracentrifugation, continuous-flow ultracentrifugation and chromatography, such as ion exchange chromatography, size exclusion chromatography, and liquid affinity chromatography. Other examples of purification methods suitable for use in the invention include polyethylene glycol or ammonium sulfate precipitation, as well as ultrafiltration and microfiltration.

The purified viral preparation of the invention is substantially free of contaminating proteins derived from the cells or cell culture and preferably comprises less than about 50 pg cellular nucleic acid/μg virus antigen. Still more preferably, the purified viral preparation comprises less than about 20 pg, and even more preferably, less than about 10 pg. Methods of measuring host cell nucleic acid levels in a viral sample are known in the art. Standardized methods approved or recommended by regulatory authorities such as the WHO or the FDA are preferred.

The invention also includes compositions comprising attenuated TTV. As used herein, attenuation refers to the decreased virulence of TTV in a porcine subject. Methods of attenuating viruses are known in the art. Such methods include serial passage of the virus in cultured cells as described above, until the virus demonstrates attenuated function. The temperature at which the virus is grown can be any temperature at which tissue culture passage attenuation occurs. Attenuated function of the virus after one or more passages in cell culture can be measured by one skilled in the art. Evidence of attenuated function may be indicated by decreased levels of viral replication or by decreased virulence in an animal model, as described above.

One particular method of producing an attenuated TTV includes passage of the virus in cell culture at suboptimal or “cold” temperatures and/or introduction of attenuating mutations into the TTV genome by random mutagenesis (e.g., chemical mutagenesis using for example 5-fluorouracil) or site specific-directed mutagenesis. Cold adaptation generally includes passage at temperatures between about 20° C. to about 32° C., and preferably between temperatures of about 22° C. to about 30° C., and most preferably between temperatures of about 24° C. and 28° C. The cold adaptation or attenuation may be performed by passage at increasingly reduced temperatures to introduce additional growth restriction mutations. The number of passages required to obtain safe, immunizing attenuated virus is dependent at least in part on the conditions employed. Periodic testing of the TTV culture for virulence and immunizing ability in animals can be used to readily determine the parameters for a particular combination of tissue culture and temperature.

TTV can also be attenuated by mutating one or more of the various viral regions, such as ORF1, ORF2 and/or ORF3, to reduce expression of the viral structural or nonstructural proteins. The attenuated TTV may comprise one or more additions, deletions or insertion in one or more of the regions of the viral genome.

Once attenuated, the virus is purified using techniques known in the art, such as described above with reference to inactivated viruses.

Subunit compositions can also be produced. For example, one or more immunogens derived from any of the viral genomic regions, such as derived from any of ORF1, ORF2 and/or ORF3, can be generated using recombinant methods, well known in the art. In this regard, oligonucleotide probes can be devised based on the sequences of the TTV genome and used to probe genomic or cDNA libraries for TTV genes encoding for the immunogens useful in the present invention. The genes can then be further isolated using standard techniques and, if desired, restriction enzymes employed to mutate the gene at desired portions of the full-length sequence. Alternatively, DNA sequences encoding the proteins of interest can be prepared synthetically rather than cloned. The DNA sequences can be designed with the appropriate codons for the particular amino acid sequence. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311. TTV genes can also be isolated directly from viruses using known techniques, such as phenol extraction, and the sequence can be further manipulated to produce any desired alterations. See, e.g., Sambrook et al., supra, for a description of techniques used to obtain and isolate DNA.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. If signal sequences are included, they can either be the native, homologous sequences, or heterologous sequences. Leader sequences can be removed by the host in post-translational processing. See, e.g., U.S. Pat. Nos. 4,431,739; 4,425,437; 4,338,397.

Other regulatory sequences may also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the sequence of interest. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sam brook et al., supra; DNA Cloning, Vols. I and II, supra; Nucleic Acid Hybridization, supra.
It is often desirable that the polypeptides prepared using the above systems are fusion polypeptides. As with nonfusion proteins, these proteins may be expressed intracellularly or may be secreted from the cell into the growth medium.

Furthermore, plasmids can be constructed which include a chimeric gene sequence, encoding e.g., multiple TTV immunogens. The gene sequences can be present in a dicistronic gene configuration. Additional control elements can be situated between the various genes for efficient translation of RNA from the distal coding region. Alternatively, a chimeric transcription unit having a single open reading frame encoding the multiple antigens can also be constructed. Either a fusion can be made to allow for the synthesis of a chimeric protein or alternatively, protein processing signals can be engineered to provide cleavage by a protease such as a signal peptidase, thus allowing liberation of the two or more proteins derived from translation of the template RNA. The processing protease may also be expressed in this system either independently or as part of a chimera with the antigen and/or cytokine coding region(s). The protease itself can be both a processing enzyme and a vaccine antigen.

The expression vector is then used to transform an appropriate host cell. The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells (e.g., HEK293), human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromycesfragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the immunogens of the present invention are produced by growing host cells transformed by an expression vector under conditions whereby the immunogen of interest is expressed. The immunogen is then isolated from the host cells and purified. If the expression system provides for secretion of the immunogen, the immunogen can be purified directly from the media. If the immunogen is not secreted, it is isolated from cell lysates. The selection of the appropriate growth conditions and recovery methods are within the skill of the art.

The TTV immunogens may also be produced by chemical synthesis such as by solid phase or solution peptide synthesis, using methods known to those skilled in the art. Chemical synthesis of peptides may be preferable if the antigen in question is relatively small. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Velag, Berlin (1984) and E. Gross and J. Meienhofer, Eds., The Peptides: Analysis, Synthesis, Biology, supra, Vol. 1, for classical solution synthesis.

The TTV immunogens can be used to produce antibodies, both polyclonal and monoclonal. If polyclonal antibodies are desired, a selected mammal, (e.g., mouse, rabbit, goat, horse, etc.) is immunized with an immunogen of the present invention, or its fragment, or a mutated immunogen. Serum from the immunized animal is collected and treated according to known procedures. See, e.g., Jurgens et al. (1985) J. Chroni. 348:363-370. If serum containing polyclonal antibodies is used, the polyclonal antibodies can be purified by immunoaffinity chromatography, using known procedures.

Monoclonal antibodies to the TTV immunogens, can also be readily produced by one skilled in the art. The general methodology for making monoclonal antibodies by using hybridoma technology is well known. Immortal antibody-producing cell lines can be created by cell fusion, and also by other techniques such as direct transformation of B lymphocytes with oncogenic DNA, or transfection with Epstein-Barr virus. See, e.g., M. Schreier et al., Hybridoma Techniques (1980); Hammerling et al., Monoclonal Antibodies and T-cell Hybridomas (1981); Kennett et al., Monoclonal Antibodies (1980); see also U.S. Pat. Nos. 4,341,761; 4,399,12 1; 4,427,783; 4,444,887; 4,452,570; 4,466,917; 4,472,500, 4,491,632; and 4,493,890. Panels of monoclonal antibodies produced against the TTV immunogen of interest, or fragment thereof, can be screened for various properties; i.e., for isotype, epitope, affinity, etc. Monoclonal antibodies are useful in purification, using immunoaffinity techniques, of the individual antigens which they are directed against. Both polyclonal and monoclonal antibodies can also be used for passive immunization or can be combined with subunit vaccine preparations to enhance the immune response.

TTV Formulations and Administration

The inactivated, attenuated or isolated TTV immunogens of the present invention can be formulated into compositions, such as vaccine or diagnostic compositions, either alone or in combination with other antigens, for use in immunizing subjects as described below. For example, the compositions can include additional immunogens from pathogens that cause disease in pigs, such as but not limited to, immunogens from porcine parvovirus, porcine circovirus, porcine reproductive and respiratory syndrome virus, swine influenza, pseudorabies virus, pestivirus which causes porcine swine fever, porcine lymphotropic herpesviruses (PLHV1 and PLHV2), Mycoplasma spp, Helicobacter spp, Campylobacter spp, Lawsonia spp, Actinobacillus pleuropneumoniae, Haemophilus parasuis, Streptococcus spp, Pasteurella spp, Salmonella spp, E. coli, Clostridium spp, Eryspelothrix rhusiopathiae.

Methods of preparing such formulations are described in, e.g., Remington's Pharmaceutical Sciences, Mack Publishing Company, Easton, Pennsylvania, 18 Edition, 1990. Typically, the vaccines of the present invention are prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. Vaccines suitable for oral delivery can also be readily formulated. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the vaccine may also be added to the formulation. Adjuvants may include for example, muramyl dipeptides, avridine, aluminum hydroxide, alum, Freund's adjuvant, incomplete Freund's adjuvant (ICFA), dimethyldioctadecyl ammonium bromide (DDA), oils, oil-in-water emulsions, saponins, cytokines, and other substances known in the art. Such adjuvants are well known and commercially available from a number of sources, e.g., Difco, Pfizer Animal Health, Newport Laboratories, etc.

TTV immunogens may also be linked to a carrier in order to increase the immunogenicity thereof. Suitable carriers include large, slowly metabolized macro-molecules such as proteins, including serum albumins, keyhole limpet hemocyanin, immunoglobulin molecules, thyroglobulin, ovalbumin, and other proteins well known to those skilled in the art; polysaccharides, such as sepharose, agarose, cellulose, cellulose beads and the like; polymeric amino acids such as polyglutamic acid, polylysine, and the like; amino acid copolymers; and inactive virus particles.

TTV immunogens may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides.

Furthermore, the TTV immunogens may be formulated into vaccine compositions in either neutral or salt forms. Pharmaceutically acceptable salts include the acid addition salts (formed with the free amino groups of the active polypeptides) and which are formed with inorganic acids such as, for example, hydrochloric or phosphoric acids, or such organic acids as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Vaccine formulations will contain a “therapeutically effective amount” of the active ingredient, that is, an amount capable of eliciting an immune response in a subject to which the composition is administered. In the treatment and prevention of TTV infection, a “therapeutically effective amount” is readily determined by one skilled in the art using standard tests. The TTV immunogens will typically range from about 1% to about 95% (w/w) of the composition, or even higher or lower if appropriate. With the present vaccine formulations, 0.1 to 500 mg of active ingredient per ml, preferably 1 to 100 mg/ml, more preferably 10 to 50 mg/ml, such as 20 . . . 25 . . . 30 . . . 35 . . . 40, etc., or any number within these stated ranges, of injected solution should be adequate to raise an immunological response when a dose of 0.25 to 3 ml per animal is administered.

If an inactivated or attenuated preparation is used, the compositions will generally include 10² to 10¹² pfu, more particularly from 10⁴ to 10⁸ pfu, and preferably from 10⁵ to 10⁷ pfu of TTV.

To immunize a subject, the vaccine is generally administered parenterally, usually by intramuscular injection. Other modes of administration, however, such as subcutaneous, intraperitoneal and intravenous injection, are also acceptable. The quantity to be administered depends on the animal to be treated, the capacity of the animal's immune system to synthesize antibodies, and the degree of protection desired. Effective dosages can be readily established by one of ordinary skill in the art through routine trials establishing dose response curves. The subject is immunized by administration of the vaccine in at least one dose, and preferably two or more doses. Moreover, the animal may be administered as many doses as is required to maintain a state of immunity to infection.

Additional vaccine formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Controlled or sustained release formulations are made by incorporating the protein into carriers or vehicles such as liposomes, nonresorbable impermeable polymers such as ethylenevinyl acetate copolymers and Hytrel copolymers, swellable polymers such as hydrogels, or resorbable polymers such as collagen and certain polyacids or polyesters such as those used to make resorbable sutures. The TTV immunogens can also be delivered using implanted mini-pumps, well known in the art.

The TTV immunogens of the instant invention can also be administered via a carrier virus which expresses the same. Carrier viruses which will find use with the instant invention include but are not limited to the vaccinia and other pox viruses, adenovirus, and herpes virus. By way of example, vaccinia virus recombinants expressing the novel proteins can be constructed as follows. The DNA encoding the particular protein is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the instant protein into the viral genome. The resulting T⁻-recombinant can be selected by culturing the cells in the presence of 5-bromodeoxyuridine and picking viral plaques resistant thereto.

An alternative route of administration involves gene therapy or nucleic acid immunization. Thus, nucleotide sequences (and accompanying regulatory elements) encoding the subject TTV immunogens can be administered directly to a subject for in vivo translation thereof. Alternatively, gene transfer can be accomplished by transfecting the subject's cells or tissues ex vivo and reintroducing the transformed material into the host. DNA can be directly introduced into the host organism, i.e., by injection (see International Publication No. WO/90/11092; and Wolff et al. (1990) Science 247:1465-1468). Liposome-mediated gene transfer can also be accomplished using known methods. See, e.g., Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219A; and Nabel et al. (1990) Science 1990) 249:1285-1288. Targeting agents, such as antibodies directed against surface antigens expressed on specific cell types, can be covalently conjugated to the liposomal surface so that the nucleic acid can be delivered to specific tissues and cells susceptible to infection.

Diagnostics

The TTV compositions, can also be used as diagnostics to detect the presence of reactive antibodies directed against TTV in a biological sample. For example, the presence of antibodies reactive with the TTV preparations can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound antibody in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g, beads or microtiter plates); polyvinylidine fluoride; diazotized paper; nylon membranes; activated beads, magnetically responsive beads, and the like.

Typically, a solid support is first reacted with a solid phase component (e.g., one or more TTV antigens, under suitable binding conditions such that the component is sufficiently immobilized to the support. Sometimes, immobilization of the antigen to the support can be enhanced by first coupling the antigen to a protein with better binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind the antigens to the support include polysaccharides, polylactic acids, polyglycolic acids, polymeric amino acids, amino acid copolymers, and the like. Such molecules and methods of coupling these molecules to the antigens, are well known to those of ordinary skill in the art. See, e.g., Brinkley, M. A., Bioconjugate Chem. (1992) 3:2-13; Hashida et al., J. Appl. Biochem. (1984) 6:56-63; and Anjaneyulu and Staros, International J. of Peptide and Protein Res. (1987) 30:117-124.

After reacting the solid support with the solid phase component, any non-immobilized solid-phase components are removed from the support by washing, and the support-bound component is then contacted with a biological sample suspected of containing ligand moieties (e.g., antibodies toward the immobilized antigens) under suitable binding conditions. After washing to remove any non-bound ligand, a secondary binder moiety is added under suitable binding conditions, where the secondary binder is capable of associating selectively with the bound ligand. The presence of the secondary binder can then be detected using techniques well known in the art.

More particularly, an ELISA method can be used, where the wells of a microtiter plate are coated with the TTV antigen(s). A biological sample containing or suspected of containing anti-TTV immunoglobulin molecules is then added to the coated wells. In assays where it is desired to use one microtiter plate, a selected number of wells can be coated with, e.g., a first antigen moiety, a different set of wells coated with a second antigen moiety, and so on. In the alternative, a series of ELISAs can be run in tandem. After a period of incubation sufficient to allow antibody binding to the immobilized antigens, the plate(s) can be washed to remove unbound moieties and a detectably labeled secondary binding molecule added. The secondary binding molecule is allowed to react with any captured sample antibodies, the plate washed and the presence of the secondary binding molecule detected using methods well known in the art.

Thus, in one particular embodiment, the presence of bound anti-TTV antigen ligands from a biological sample can be readily detected using a secondary binder comprising an antibody directed against the antibody ligands. A number of useful immunoglobulin (Ig) molecules are known in the art and commercially available. Ig molecules for use herein will preferably be of the IgG or IgA type, however, IgM may also be appropriate in some instances. The Ig molecules can be readily conjugated to a detectable enzyme label, such as horseradish peroxidase, glucose oxidase, Beta-galactosidase, alkaline phosphatase and urease, among others, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the viral proteins and antibodies specific for those viral proteins form complexes under precipitating conditions. In one particular embodiment, the TTV antigen(s) can be attached to a solid phase particle (e.g., an agarose bead or the like) using coupling techniques known in the art, such as by direct chemical or indirect coupling. The antigen-coated particle is then contacted under suitable binding conditions with a biological sample suspected of containing antibodies for TTV. Cross-linking between bound antibodies causes the formation of particle-antigen-antibody complex aggregates which can be precipitated and separated from the sample using washing and/or centrifugation. The reaction mixture can be analyzed to determine the presence or absence of antibody-antigen complexes using any of a number of standard methods, such as those immunodiagnostic methods described above.

In yet a further embodiment, an immunoaffinity matrix can be provided, wherein a polyclonal population of antibodies from a biological sample suspected of containing anti-TTV antibodies is immobilized to a substrate. In this regard, an initial affinity purification of the sample can be carried out using immobilized antigens. The resultant sample preparation will thus only contain anti-TTV moieties, avoiding potential nonspecific binding properties in the affinity support. A number of methods of immobilizing immunoglobulins (either intact or in specific fragments) at high yield and having good retention of antigen binding activity, are known in the art. Not being limited by any particular method, immobilized protein A or protein G can be used to immobilize immunoglobulins.

Accordingly, once the immunoglobulin molecules have been immobilized to provide an immunoaffinity matrix, the TTV antigens, having separate and distinct labels, are contacted with the bound antibodies under suitable binding conditions. After any non-specifically bound antigen has been washed from the immunoaffinity support, the presence of bound antigen can be determined by assaying for each specific label using methods known in the art.

The above-described assay reagents, including the TTV immunogens, optionally immobilized on a solid support, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Transmission of Porcine Genogroup 1 TTV to Gnotobiotic Swine

In order to determine the ability of TTV to infect gnotobiotic swine in order to produce a porcine model of TTV, the following experiment was conducted.

### MATERIALS AND METHODS

### Animals:

Gnotobiotic swine were derived by Caesarian section from date-mated gravid sows; animal husbandry conditions and sampling procedures for periodic collections of blood samples have been reported previously (Krakowka S, Eaton K A (1996) in Advances in Swine in Biomedical Research II, eds Tumbleson M, Schook L (Plenum Press, NY), pp 779-810).

### Source of Infectious TTV and PCR Assays:

Aseptic citrated blood and serum samples were collected from 20 healthy young (14-16 week old) swine from a specific pathogen-free herd and kept on ice until transported to the laboratory. The leukocyte-rich plasmas were collected by low speed centrifugation and individually stored at −70C. Companion sera were tested for porcine circovirus 2 (PCV2) DNAs by PCR as described (McIntosh et al., Can. J. Vet. Res. (2006) 70:58-61) and also for porcine genogroups (g) 1 and g2-TTV DNAs with a nPCR assay using published primer sequences for swine (Kekarainen et al., J. Gen. Virol. (2006) 87:833-837).

### Amplified Sequence Analysis:

The nPCR-positive samples were expanded and sequenced by routine methods. The sequences obtained were then aligned with porcine genogroups (g) 1 and 2 TTV DNA sequences stored in Genbank and identified as either porcine g1 or g2 by homology to these published sequences.

### Serial Passage in Gnotobiotic Swine:

The PCV2-DNA negative, TTV-positive leukocyte-rich plasmas (n=18) were pooled and inoculated intraperitoneally (IP) into three 3-day old gnotobiotic piglets (8.5 ml/piglet). Twenty-eight days after inoculation, two piglets were TTV DNA-viremia positive by nPCR. These piglets were terminated and a 20% (w/v) liver homogenate was made from one piglet and frozen at −70C. This first passage (p) homogenate was thawed and subjected to two rounds of extraction with chloroform (c) to remove infectivity of any extraneous enveloped viruses contained in the pl homogenate (Feinstone et al., Infect. Immun. (1983) 41:816-821). Five ml of the freshly thawed cTTVp1 homogenate was given IP to two 2-day-old gnotobiotic piglets; these piglets were terminated on post infection day (PID 10). The livers and bone marrows from these piglets were aseptically collected and tested separately for TTV by nPCR. A 10% (w/v) bone marrow-liver homogenate was prepared from the piglet with the strongest nPCR TTV signal, treated twice with chloroform and the aqueous phase (cTTVp2) was frozen (−70C). The cTTVp2 homogenate was thawed and 5.0 ml was inoculated IP into two 15-day-old gnotobiotic piglets as above and the livers and bone marrows collected from each piglet after termination on PID 10 (cTTVp3). As before, bone marrows and livers were tested separately for TTV DNAs by PCR and nPCR and the paired BM/liver samples with the most prominent primer pair binding band were homogenized in phosphate buffered saline as a 10% (w/v) suspension to create the fourth in vivo passed TTV (TTVp4). The fourth pass (TTVp4) was not extracted with chloroform but instead divided into 2.0 ml aliquots and frozen (−70C) as stock infectious virus for subsequent in vivo pathogenesis studies.

### Histology and Immunohistochemistry (IHC):

At termination, tissue sections from all TTV-inoculated piglets and controls (inguinal, axillary, mesenteric and bronchial lymph nodes, thymus, bone marrow, spleen, liver, lung, kidney and ileum) were collected into cold (4C) ethanol, fixed for 24 hrs and then processed for histologic examination and staining by hematoxylin and eosin using routine methods. Tissue section replicates were stained for PCV2 nucleocapsid protein as described (Krakowka et al., Vet. Pathol. (2001) 38:31-42; Krakowka et al., Can. Vet. J. (2007) 48:716-724). Section replicates of kidney were stained with the Jones silver and periodic acid Schiff (PAS) stains for basement membranes, porcine fibrinogen/fibrin (clone MFB-HB, Accurate Chemical & Scientific Corporation, Westbury, N.Y.) followed by biotinylated equine anti-mouse IgG, developed with the Standard ABC kit (Vectastain, Vector Laboratories, Burlingame, Calif.) and diamino benzidine (DAB) peroxidase substrate. Selected renal section replicates were stained for deposits of porcine IgG using goat anti-porcine IgG followed by biotinylated anti-goat IgG, (Kirkegaard and Perry Laboratories, Gaithersburg, Md.) developed with the Standard ABC kit and diamino benzidine.

### In Situ Hybridization:

Selected tissues were tested for the presence of TTV DNA by in situ hybridization (ISH) using designed g1-TTV primers and digoxin-labeled nucleotides. Briefly, ethanol-fixed tissue section replicates were de-waxed with xylene, quenched with hydrogen peroxide in methanol, digested with protease and hybridized with labeled probe (37 and 55C) by standard methods. Labeled probe was detected using a monoclonal anti-digoxin antibody coupled to horseradish peroxidase and developed with diamino benzidine essentially as described elsewhere (Hamberg et al., J. Vet. Diag. Invest. (2007) 19:135-141). Controls for ISH consisted of ISH using a PCV2-specific probe, ISH with omission of the monoclonal anti-digoxin antibody, staining tissues with the monoclonal without prior ISH and ISH on TTV-negative tissue sections.

### Screening for Porcine Pathogens:

All terminal sera, including the samples collected from the TTV-positive conventional pigs prior to the transmission experiments as well as subsequent tissue homogenate TTV pools and terminal serum samples from gnotobiotic swine were tested for the presence of porcine parvovirus (PPV), swine influenza virus (SIV), porcine respiratory and reproductive syndrome virus (PRRSV) by reverse transcriptase (rt) PCR for PRRSV and SIV RNAs porcine circovirus (PCV) types 1 and 2 by PCR. Convalescent sera were also tested for antibodies to these viruses by virus neutralization (encephalomyocarditis virus and transmissible gastroenteritis virus), agar gel immunoprecipitation, (SIV), hemagglutination inhibition assay (PPV) and ELISA (PRRSV).

Experimental design:

Gnotobiotic piglets were inoculated with 2.0 ml of the chloroform-extracted first passage TTV (cTTVp1) intraperitoneally (IP) at 2 days of age and terminated on PIDs 5 (n−2), 7 (n=2), 21 (n=2) and 34 (n=3) for gross and histologic evaluations. Uninfected control separately housed piglets from the same litter (n=2) were not infected and were terminated on PIDs 6 and 34 respectively. In a second in vivo challenge experiment, piglets from two gnotobiotic litters (n=11) were similarly inoculated IP with 2.0 ml of TTVp4 at 2 days of age and terminated on PIDs 3 (n=1), 5 (n=2), 7 (n=1), 14 (n=3) and 35 (n=2). Two separately housed littermates were kept as uninfected controls and were terminated at 34 days of age.

### RESULTS

### Amplicon(s) Detected by Direct and Nested PCR:

Both the nested and direct PCR assays generated an amplicon of 292 bases from the pooled plasmas of the initial inoculum and in the serum, liver and bone marrows of piglets inoculated with cTTVp1 through cTTVp3 and TTVp4. Sequences recovered from cTTVp1 and TTVp4 were amplified, sequenced and compared to sequences reported in Genbank for porcine TTV reference genogroup (g) 1 (DQ229865) and g2 (DQ229860). Both the cTTVp1 and TTVp4 amplicons were matched to the UTR of the genome; each was 94.3% homologous to g1-TTV and less than 45% sequence-identical to g2-TTV. Genogroup 2 TTV DNAs were not found in any of the piglets inoculated with cTTVp1 through TTVp4. By quantitative PCR (qPCR), the number of copies of g1-TTV DNA per nanogram (ng) of extracted total DNA was determined for the inocula developed for each in vivo passage. Values were calculated as follows: cTTVp1 (<10 copies/ng DNA), cTTVp2 (2.0×10³ copies/ng DNA), cTTVp3 (1.6×10⁴ copies/ng DNA) and TTVp4 (1.1×10⁴ copies/ng DNA).

### Clinical Signs and Gross Findings:

No clinical signs were observed in any of the cTTVp1- or the TTVp4-infected gnotobiotic piglets. Interstitial pneumonia was identified by gross examination of tissues in two of three piglets terminated on PID 5, one of three piglets terminated on PID 7 and two of three piglets terminated on 14. Mild thymic atrophy was seen in piglets terminated on PIDs 3 through 7. Two piglets terminated on PID 14 had focal edema in the ventral neck and thoracic mediastinum. One PID 21 piglet exhibited local edema in the right axillary lymph node and brachial plexus region. Piglets terminated on PID 34 were grossly normal. No gross lesions were seen in the uninfected normal gnotobiotic swine.

Histologic findings:

All lymphoid tissues, regardless of infectious status or PID were hypoplastic and inactive; rare germinal centers were seen in bronchial and mesenteric lymph nodes of both infected and control piglets, most likely related to mucosal antigen (sow milk replacement formula) stimulation through the diet. The thymuses of piglets terminated before PID 7 were variably reduced in size, chiefly due to reduction of the T lymphocyte-rich thymic cortices. This change was transient (thymuses from PID 14 onward were indistinguishable from controls) and the reduction in thickness of the cortex was not associated with obvious cellular necrosis or inflammatory changes in the cortex. The liver of all piglets terminated by PID 21 or sooner had vacuolar cytoplasmic changes in hepatocytes compatible with mild fatty degeneration/glycogen infiltration. Foci of extramedullary hematopoiesis were present in both control and infected piglets through day 10 of age (PID 7). Small foci of mixed (lymphocytes and histiocytes) were present in PID 14 livers onward.

Time (PID dependent) histologic changes were most dramatic in the lungs of g1-TTV-infected piglets. Alveolar septal walls in the PID 5-7 piglets were thickened by a modest, infiltration with mononuclear cells and extracellular proteinic material. Fibrinous exudation into the alveolar air spaces was present in the pulmonary sections prepared from one PID 7 piglet. One of three PID 14 (and 1 of 4 PID 34 piglets as well) piglets had cellular debris admixed with fibrin in alveolar spaces. By PIDs 14-21, pulmonary changes had matured into an obvious diffuse interstitial pneumonia characterized by accumulation of proteinic material in capillary lumens and a diffuse lymphocytic and histiocytic cellular infiltrate into the interstitium. Diffuse and lobular interstitial pneumonia was still present in three of four piglets terminated on PID 34. In these pigs, the pulmonary lesions were less severe than that observed in the PID 21 piglets except for a regionally extensive area fibrinous alveolitis and interstitial pneumonia, associated with activated mononuclear inflammatory cells and syncytial giant cells identified in one piglet.

No histologic changes were seen in the kidneys from g1-TTV-infected piglets terminated before PID 7. However, in the kidneys of g1-TTvp4-infected piglets on PID 14, renal glomeruli were variably distended with eosinophilic extracellular proteinic material with occasional inflammatory cells entrapped in the glomerular spaces. The renal glomerular lesion progressed by PID 21 and was still present on PID 34 at which time selected glomeruli has become scarred with fibrous connective tissue proliferation within them. The morphologic changes in renal glomeruli were compatible with a provisional morphologic diagnosis of membranous glomerulonephropathy. This glomerular lesion was not present in the uninfected controls. Theses glomerular lesions were PAS-positive, exhibited irregular nodular thickening of basement membranes with the Jones silver stain and were IHC-positive for porcine fibrinogen, fibrin and IgG.

### In Situ Hybridization (Ish):

Direct visualization of g1-TTV DNAs was accomplished in selected tissues by ISH. Bone marrow samples from PID 5-infected piglets contained positive signal. The DAB reaction product was confined to the cytoplasm and rare intranuclear locations within large monocytoid-like cells of undetermined lineage. Reaction product occurred as diffuse cytoplasmic staining or as discrete cytoplasmic granules suggestive of viral inclusion bodies. Similar cells containing TTV DNAs were found in the PID 5 spleen as well.

### Other Porcine Pathogens:

All piglets were sero-negative for porcine respiratory and reproductive virus (PRRSV) by ELISA and all terminal sera were negative for PRRSV RNA by rtPCR. Terminal sera from piglets were PCV2 DNA-negative by PCR and tissue section replicates stained for PCV2 nucleocapsid protein were uniformly negative. Terminal sera were negative for swine influenza virus (SIV) antibodies and were porcine parvovirus HA1-negative.

### TTV PCR:

The various TTV tissue homogenates and all of the piglets inoculated with these homogenates were positive by nPCR or direct PCR for g1-TTV. In all cases, pre-inoculation gnotobiotic piglet sera were TTV-DNA negative. Piglets inoculated with cTTVp2 and beyond, contained sufficient TTV DNAs in tissues such that a conventional PCR assay was sufficient to detect TTV DNAs. Genogroup 1 viral DNAs were present in every tissue examined (sera, lymph nodes, thymus, lungs, liver, kidney, spleen and bone marrow) after PID 10 (data not shown).

To summarize, in the transmission experiments described above, g1-TTV DNAs were identified in the sera of all 20 conventional pigs used as the source material of TTV for in vivo passage through gnotobiotic swine. TTV was readily transmitted to young TTV DNA-negative gnotobiotic swine with TTV-positive pooled plasma and serial passage increased the amount of TTV DNAs in these piglets such that a nested PCR assay for detection of TTV DNA was no longer needed after in vivo passage 3. After PID 10, TTV DNAs were detected in all tissues. By ISH, TTV DNA was first identified in bone marrow cells, a location identified as a site of human TTV replication in vivo (Kikuchi et al., J. Med. Virol. (2000) 61:165-170;Okamoto et al., Biochem. Biophys. Res. Commun. (2002) 270:657-662; Rodriguez-Inigo et al., Am. J. Pathol. (2000) 156:1227-1234).

The spectrum of histologic lesions associated with TTV infection in gnotobiotic swine exhibited PID-dependent increased severity then reduction during convalescence (after PID 21). The lesions were reproducible and absent in age-matched uninfected control littermates; TTV-associated histologic changes were not associated with clinical signs of disease. In children, TTV infection is associated with respiratory disease and asthma (Matti et al., J. Virol. (2003) 77:2418-2425; Biagini et al., Clin. Infect. Dis. (2003) 36:128-129; Pifferi et al., J. Infect. Dis. (2005) 192:1141-1148). The interstitial pneumonia that developed in TTV-infected swine was detected by gross examination and confirmed by histology strongly suggests that swine TTV infection also causes sub-clinical respiratory disease. Similarly, lymphocytic-histiocytic cellular infiltrates in the liver of TTV-infected gnotobiotes suggest that the liver may be a site of TTV replication. Indeed in humans, TTV replicative isoforms (double stranded DNAs) have been demonstrated in liver homogenates and TTV DNAs have been identified in the cytoplasm of hepatocytes by in situ hybridization (Rodriguez-Inigo et al., Am. J. Pathol. (2000) 156: 1227-1234).

The renal glomerular lesions identified in TTV-infected gnotobiotes were distinctive. Membranous glomerulonephropathy may be associated with circulating TTV immune complexes. In humans infected with TTV, various authors have suggested that glomerular lesions may be associated with TTV infection (Usta et al., Clin. Nephrol. (2001) 55:335). In this experiment, renal glomeruli were variably thickened as demonstrated by the Jones silver stain for basement membranes, were PAS positive and contained discontinuous aggregates of porcine fibrin/fibrinogen and IgG in or on them. That these changes were absent on PIDs 5/7 but present by PID 14-21 strongly suggest that they are related to TTV viremia and developing anti-TTV antibody responses in infected gnotobiotes.

One of the hallmarks of gnotobiology is systemic underdevelopment of lymph nodes and related lymphoid aggregates. The B cell-associated germinal centers are largely absent, the para-cortical T cell-rich zones are underdeveloped and subcapsular sinuses are devoid of lymphocytes. As a result, gnotobiotes are lymphopenic and hypogammaglobulinemic (Krakowka S, Eaton K A (1996) in Advances in Swine in Biomedical Research II, eds Tumbleson M, Schook L (Plenum Press, NY), pp 779-810). However, lymphoid tissues in gnotobiotes are reactive to infectious diseases such as infection with PCV2 (Krakowka et al., Vet. Pathol. (2001) 38:3142), porcine parvovirus (Krakowka et al., Vet. Pathol. (2000) 37:254-266) and Helicobacter species (Krakowka et al., Infect. Immun. (1987) 55:2789-2796; Krakowka et al., Vet. Pathol. (1998) 35:274-282; Krakowka et al., Am. J. Vet. Res. (2005) 66:945-952). Infection with these agents results in prompt lymphoid activation manifest as lymphoid tissue hyperplasia and lymphoid follicle development; the full complement of immune responses occur in pathogen-infected gnotobiotes. That histologic changes in lymphoid tissues, aside from transient thymic atrophy, were not observed in TTV-infected swine is surprising. Regardless, histologic lesions in the lungs, liver and kidneys of TTV-infected pigs are clearly associated with TTV infection. Importantly, these changes, had they occurred in conventional swine, would not be diagnostic for TTV (or any other porcine commensal or pathogenic microbe for that matter) and would likely be dismissed as background histologic changes associated with subclinical responses to a variety of unidentified environmental insults. However, as uninfected gnotobiotic pigs lack these changes in not only this series but also as reported previously (Krakowka S, Eaton K A (1996) in Advances in Swine in Biomedical Research II, eds Tumbleson M, Schook L (Plenum Press, NY), pp 779-810), gnotobiotic conditions permit an opportunity to identify subtle histologic changes associated with TTV infection.

The implications of these findings for porcine TTVs are of great significance to the medical community. Since their discovery in 1997, investigators have repeatedly tried to implicate the TTVs in a variety of human disease processes. All of the previous studies were confounded by the high incidence of asymptomatic TTV infection in age-matched cohort controls and by the fact that the earliest phases of TTV infection cannot be identified in humans except in those rare instances of mother-to-fetus transmission or perhaps by prospective studies of patients who receive TTV-positive blood and blood products. Further, lack of suitable animal models of TTV infection has hindered investigations into the potential pathogenicity of these agents for humans. Transmission of human-origin TTVs to primates has been accomplished (Tawara et al., Biochem. Biophys. Res. Commun. (2000) 278:470-476), but these experiments are complicated by the fact that nonhuman primates have their own simian TTVs.

Thus, gnotobiotics represent a new approach to the study of the TTVs. By using gnotobiotic animals, environmental influences can be excluded, including inadvertent transmission of TTVs contained in the environment to the experimental subjects. Gross and histologic changes observed in pathogen-infected gnotobiotes are direct reflections of the interactions between the agent and the host and, by definition, are not confounded by external variables such as commensal and concurrent infections, dietary differences and environmental pressures. Leads developed in this model system may have broad implications for the study of human TTVs.

### EXAMPLE 2

### Potentiation of PCV2-Associated PMWS by Co-Infection with Porcine Genogroup 1 TTV

### MATERIALS AND METHODS

### Gnotobiotic Swine:

A total of 36 gnotobiotic piglets were derived from five date-mated pregnant sows by Caesarian section and held in sterile pen-tub isolation units (3-6 piglets per unit). Each infection group was housed in separate self-contained isolation units to maintain biosecurity and fed a commercial sow milk replacement diet as described (Krakowka S, Eaton K A (1996) in Advances in Swine in Biomedical Research II, eds Tumbleson M, Schook L (Plenum Press, NY), pp 779-810). Prior to and at the conclusion of the experiments, each isolation unit (food, feces and cages) was cultured for aerobic and anaerobic bacterial growth to confirm the gnotobiotic status; all cultures were negative for bacterial colonies.

### Viruses:

The fourth in vivo pass of Stoon 1010 strain PCV2 (OSU/PCV2p4) was used as the source of PCV2 challenge inoculum. This 10% (w/v) tissue pool contained 5×10⁸ PCV2 infectious units per ml and is free of genogroups 1 and 2 TTV DNAs as determined by nested PCR assay (Kekarainen et al., J. Gen. Virol. (2006) 87:833-837). Porcine genogroup 1 TTV was initially recovered in gnotobiotic swine by transfusion of TTV DNA-positive plasma from young conventional swine into neonatal gnotobiotic piglets as described above. TTV DNA-positive liver and bone marrow homogenates were extracted with chloroform (Feinstone et al., Infect. Immun. (1983) 41:816-821) to eliminate all enveloped viral pathogens (TTVp1) and then serially passed through three additional generations of gnotobiotic piglets, using chloroform extraction between passes 1 through 3 (TTVp1-3 respectively). A 10% (w/v) homogenate of pass 4 homogenate (TTVp4) was not extracted with chloroform, divided into 2.0 ml amounts and frozen (−70C) for subsequent in vivo challenge experiments.

### Experimental Design:

The piglets were divided into infection groups as follows: Group A: Uninfected control piglets from 3 litters (n=5), Group B: oronasally (ON) infected with PCV2/OSUp4 alone (n=6), Group C: intraperitoneally (IP) infected with g1-TTVp1 (n=4) or g1-TTVp4 (n=4), Group D: Infected with g1-TTVp1 and PCV2/OSDUp4, 7 days later (n=6), Group E: Infected with g1-TTVp4 and OSU/PCV2, 7 days later (n=6) and Group F: Infected with PCV2/OSUp4 and g1-TTVp4, IP 7 days later. Pre-infection and weekly blood samples were collected for serum and the piglets were observed for signs of illness at least three times per day. Piglets were terminated when moribund or on post-infection days (PID) 32-34.

### PCR:

DNA from weekly or terminal serum samples was extracted from frozen (−70C) sera using conventional procedures. For routine screening of samples for TTV DNAs, a nested (n) PCR assay was employed using published primer sequences for genogroup 1 TTV as described above. Assessment of PCV2 viremia was accomplished by PCR (Mcintosh et al., Can. J. Vet. Res. (2006) 70:58-61).

### Histopathology and Immunocytochemistry (IHC):

Samples of lung, liver, kidney, ileum, spleen, thymus, superficial inguinal, axillary, bronchial and mesenteric lymph nodes were collected into cold (4C) 100% ethanol, fixed for 24 hours at 4C and then processed for histology by routine methods. Tissue section replicates were stained with hematoxylin and eosin, for PCV2 nucleocapsid protein (Krakowka et al., Vet. Pathol. (2000) 37:274-282; Krakowka et al., Vet. Pathol. (2001) 38:3142; Krakowka et al., Virol. Immunol. (2002) 15:567-582). Section replicates of the kidney were also stained for deposits of porcine fibrinogen/fibrin and porcine IgG using commercially available monoclonal antibodies to these proteins by routine methods using the Vectastain™ kit technology. Briefly deparaffinized section replicates were quenched for endogenous peroxidase with 3% (v/v) H₂O₂ in PBS, reacted with the appropriate monoclonal antibody, blocked with 4% (v/v) heat inactivated horse serum, reacted with biotinylated horse anti-mouse IgG, reacted with avidin-peroxidase and visualized with diamine benzidine (DAB).

### RESULTS

### Group A: Uninfected Control Piglets:

All uninfected control piglets of Group A were clinically normal at termination and histologic changes in tissues were compatible with previous reports (Ellis et al., J. Vet. Diagn. Invest. (1999) 11:3-14; Krakowka S, Eaton K A (1996) in Advances in Swine in Biomedical Research II, eds Tumbleson M, Schook L (Plenum Press, NY), pp 779-810; Krakowka et al., Vet. Pathol. (2000) 37:274-282; Krakowka et al., Vet. Pathol. (2001) 38:31-42; Krakowka et al., Virol. Immunol. (2002) 15:567-582). Tissue section replicates were negative for PCV2 nucleocapsid protein and pre-inoculation and terminal sera were negative for both PCV2 and g1-TTV DNAs.

### Group B: PCV2/OSUp4 Alone Piglets:

All 6 piglets infected with PCV2/OSUp4 alone survived viral challenge and all were clinically healthy, but viremic at termination. In this group, a modest bronchial lymphadenopathy was seen in 5 of 6 piglets and all 6 had mild generalized lymphadenopathy. One of 6 piglets had a pale slightly yellow liver and 4 of 6 had mild thymic atrophy. Histologically, lymphoid hyperplasia (germinal centers, population of T cell-dependent areas and modest RE hyperplasia) accounted for the lymphadenopathy seen. A few syncytial giant cells were seen in a lymph node of 1 piglet and all exhibited modest mononuclear inflammatory cell infiltrates into the liver. Tissue section replicates stained for PCV2 nucleocapsid protein revealed scattered small foci or single mononuclear cells that contained reaction product. These foci were restricted to lymphoid tissues or inflammatory cell infiltrates in the liver and lung but were most intense in bronchial lymph nodes.

Group C: g1-TTVp1 or g1-TTVp4 Alone Piglets:

Four piglets infected with g1-TTVp1 alone remained clinically healthy and were terminated on PID 32. By histology, the lymphoid tissues were hypoplastic with the only histologic changes being occasional germinal center formation in bronchial and mesenteric lymph nodes similar to the uninfected control piglets of Group A above. Three of four had occasional inflammatory cell infiltrates in liver parenchyma and diffuse interstitial pneumonia. In the kidneys, renal glomeruli were modestly distended with eosinophilic extracellular proteinic protein and segmental glomerular sclerosis. Three piglets were strongly positive by g1-TTV DNA-nPCR on PID 20 whereas one piglet was trace g1-TTV DNA positive at this time interval. Terminal serum samples from all 4 piglets were PCV2 DNA-negative by PCR. Similar results were seen in the gnotobiotic piglets infected with g1-TTVp4 alone except that the renal glomerular lesions were more severe, the interstitial pneumonia was more pronounced and all four were strongly g1-TTV DNA viremia-positive at termination. These inoculates were also clinically asymptomatic at termination. Terminal sera from all g1-TTV-infected piglets were negative for PCV2 DNAs.

Group D: g1-TTVp1 and PCV2/OSDUp4, 7 Days Later:

Three of 6 dually infected piglets developed acute, fatal and fulminant PMWS. One piglet died suddenly, 18 days after infection with PCV2 and two more piglets became moribund and were terminated 19 days after infection with PCV2. All three PMWS-affected piglets had generalized lymphadenopathy, thymic atrophy, ascites and small pale-to-yellow livers. Profound lymphoid depletion with replacement by histiocytes and sheets of granulomatous inflammation had completely replaced all remnants of B and T cell zones in lymphoid tissues. Massive and diffuse hepatocellular necrosis was present in all three piglets. Section replicates stained for PCV2 nucleocapsid protein revealed overwhelming and diffuse infection of hepatocytes and other epithelia such as bronchial epithelium, tonsilar epithelium and enterocytes overlying Peyer's patches in the ileum. In the kidney sections, aside from tubular epithelial virus-positive cells, the renal glomeruli demonstrated strong linear deposits of PCV2-positive materials of the immune complex-type morphology. These glomerular deposits were also IgG- and fibrin/fibrinogen-positive.

Gross lesions in the 3 dually infected piglets that survived the experimental period were typical of “moderate to severe” subclinical PCV2 infection. A generalized lymphadenopathy was present but all 3 thymi were within normal gross limits. In one pig there was mild ascites and in another, a slightly “pale” liver. In all subclinically infected pigs, the lungs remained inflated and firm and somewhat “firmer” than usual, suggestive of interstitial pneumonia. Two piglets had healed small renal cortical infarcts. Histology and IHC examinations revealed that all three pigs were subclinically but actively infected with PCV2. Terminal sera from all 6 piglets contained g1-TTV and PCV2 DNAs.

Group E: Infected with g1-TTVp4 and OSU/PCV2, 7 Days Later:

Six piglets were infected with TTVp4 IP at 3 days of age and one week later inoculated ON with PCV2/OSUp4. On PID 18 after PCV2 infection, 1 piglet developed sudden onset anorexia and was terminated with clinically evident PMWS (icterus, edema, ascites) on PID 19. A second piglet died suddenly on PID 25 after PCV2 infection. In this piglet, extensive pleural effusion and pulmonary edema associated with severe diffuse interstitial pneumonia was the proximate cause of death. A third piglet developed subcutaneous edema on PID 21 and anorexia and was terminated on 32 days after PCV2 infection. All 3 dead piglets had generalized lymphadenopathy, thymic atrophy and small pale yellow livers, typical of PMWS in gnotobiotic swine. Three of the 6 piglets in this inoculation group were still alive at termination at 35 days of age although one of these had mild subcutaneous edema and ascites at termination. The remaining two dually infected piglets were clinically healthy at termination and had gross and histologic lesions compatible with subclinical PCV2 infection. Histologic and IHC findings in the tissues from the pigs of this second experiment were similar to those identified in Group D except that the piglet with pleural effusion had extensive interstitial PCV2 nucleocapsid-protein-positive pneumonia.

Group F: Infected with PCV2/OSUp4 and g1-TTVp4, IP 7 Days Later:

In this last experiment, 6 gnotobiotic piglets were infected with PCV2/OSUp4 at 3 days of age and with g1-TTVp1 one week later. In addition, two piglets in this infection group were also immunostimulated by immunizations with keyhole limpet hemocyanin emulsified in incomplete Freund's adjuvant (KLH/ICFA) as done previously (Krakowka et al., Vet. Pathol. (2001) 38:31-42). Both of the KLH/ICFA-immunized piglets demonstrated a transient (2-3 days) period of anorexia and subcutaneous edema roughly 2 weeks after infection with PCV2/OSUp4 but recovered and were clinically normal at termination (35 days of age, 32 days after infection with PCV2). All six piglets survived dual infection by this infection schedule. The gross and histologic lesions in all 6 piglets were compatible with stable sub-clinical PCV2 infection in that the thymuses were of normal size, the lymphadenopathy detected was attributable to the combination of KLH/ICFA-associated granulomas and lymphoreticular hyperplasia. By IHC, the viral antigen was restricted to lymphoid tissues as single-positive cells.

To summarize, the experiments above show that active infection with porcine g1-TTV facilitates the development of PMWS in PCV2-challenged gnotobiotic piglets. While these data now add the TTVs to the group of infectious agents that promote PMWS, the mechanism(s) of TTV promotion appear to differ from that seen in other co-infection models of PMWS. In the latter, immunostimulation is regarded as central to promotion of PMWS. In the former, it appears that minimal to no histologic evidence for immunologic activation accompanies TTV infection, yet co-infection with this agent clearly promotes PMWS.

Moreover, the sequence of co-infection determines clinical outcome. TTV infection after PCV2 infection is established did not potentiate PMWS whereas TTV infection prior to infection with PCV2 did. There are other differences as well. While the latency “period” varies with either the PPV or the immunization models of PMWS, clinical expression is seen between 4 and 5 weeks after infection with PCV2. In the case of TTV, PMWS was expressed as early as PID 18 (mean PID 23), roughly two weeks earlier. Additionally, rapid expansion of PCV2 tropisms occurred in the TTV model such that significant infection of epithelia (enteric, respiratory, renal tubular and hepatic epithelial cell types) accompanied generalized lymphoid tissue infection. In this regard, the TTV model is more similar to the immunosuppressive effects model of cyclosporine-induced PMWS (Krakowka et al., Virol. Immunol. (2002) 15:567-582).

The potentiating effects mediated by g1-TTV were unexpected and certainly could not be predicted given the spectrum of histologic lesions associated with TTV infection alone. Thus, it does not appear that TTVs are in fact harmless as most investigators have assumed.

Thus, methods for treating, preventing and diagnosing TTV infection are described, as well as compositions for use with the methods. Although preferred embodiments of the subject invention have been described in some detail, it is understood that obvious variations can be made without departing from the spirit and the scope of the invention as defined by the claims.

1. A composition comprising a pharmaceutically acceptable vehicle and at least one porcine Torque teno virus (TTV) immunogen selected from the group consisting of an inactivated immunogenic porcine TTV, an attenuated immunogenic porcine TTV and an isolated immunogenic porcine TTV polypeptide.

2. The composition of claim 1, wherein the composition comprises an inactivated immunogenic porcine TTV.

3. The composition of claim 1, wherein the composition comprises an attenuated immunogenic porcine TTV.

4. The composition of claim 1, wherein the composition comprises an isolated immunogenic porcine TTV polypeptide.

5. The composition of claim 1, further comprising an adjuvant.

6. A method of treating or preventing TTV infection in a porcine subject comprising administering to said subject a therapeutically effective amount of a composition according to claim 1.

7. A method of treating or preventing a porcine circovirus-associated disease (PCVAD) in a porcine subject comprising administering to said subject a therapeutically effective amount of a composition according to claim 1.

8. The method of claim 7, wherein the PCVAD is postweaning multisystemic wasting syndrome (PMWS).

9. The method of claim 7, wherein the PCVAD is porcine dermatitis and nephropathy syndrome (PDNS).

10. The method of claim 7, wherein the PCVAD is porcine circovirus type 2 (PCV-2).

11. A method of producing a composition comprising:
(a) providing at least one porcine Torque teno virus (TTV) immunogen selected from the group consisting of an inactivated immunogenic porcine TTV, an attenuated immunogenic porcine TTV and an isolated immunogenic porcine TTV polypeptide; and
(b) combining said TTV immunogen with a pharmaceutically acceptable vehicle.

12. The method of claim 11, further comprising providing an adjuvant.

13. A method of detecting Torque teno virus (TTV) infection in a vertebrate subject comprising:
(a) providing a biological sample from the subject; and
(b) reacting said biological sample with at least one isolated immunogenic porcine TTV polypeptide, under conditions which allow TTV antibodies, when present in the biological sample, to bind with said TTV polypeptide, thereby detecting the presence or absence of TTV infection in the subject.

14. The method of claim 13, further comprising:
(c) removing unbound antibodies;
(d) providing one or more moieties capable of associating with said bound antibodies; and
(e) detecting the presence or absence of said one or more moieties, thereby defecting the presence or absence of TTV infection.

15. The method of claim 14, wherein the detectable label is a fluorescer or an enzyme.

16. The method of claim 13, wherein said biological sample is a porcine serum sample.

17. A method for infecting a young TTV-negative piglet, a barrier-raised specific pathogen-free piglet, or caesarian-delivered piglet with a porcine isolate of Torque teno virus (TTV), said method comprising:
(a) isolating TTV from a porcine subject; and
(b) administering a dose of the TTV isolate to said piglet in an amount sufficient to cause TTV infection.

18. A method for evaluating the ability of a vaccine to prevent Torque teno virus (TTV) infection comprising:
(a) administering to a porcine subject a candidate vaccine;
(b) exposing the porcine subject from step (a) to a porcine TTV isolate in an amount sufficient to cause infection in an unvaccinated subject; and
(c) observing the incidence of TTV infection in the porcine subject, thereby evaluating the ability of the candidate vaccine to prevent TTV infection.

19. The method of claim 18, wherein the porcine subject is a young TTV-negative piglet, a barrier-raised specific pathogen-free piglet, or caesarian-delivered piglet.

20. A method for evaluating the ability of a vaccine to prevent a porcine circovirus-associated disease (PCVAD) comprising:
(a) administering to a porcine subject a candidate vaccine;
(b) exposing the porcine subject from step (a) to a porcine Torque teno virus (TTV) isolate in an amount sufficient to cause infection in an unvaccinated subject; and
(c) observing the incidence of a PCVAD in the porcine subject, thereby evaluating the ability of the candidate vaccine to prevent PCVAD.

21. The method of claim 20, wherein the porcine subject is a young TTV-negative piglet, a barrier-raised specific pathogen-free piglet, or caesarian-delivered piglet.

22. The method of claim 20, wherein the PCVAD is postweaning multisystemic wasting syndrome (PMWS).

23. The method of claim 20, wherein the PCVAD is porcine dermatitis and nephropathy syndrome (PDNS).

24. The method of claim 20, wherein the PCVAD is porcine circovirus type 2 (PCV-2).

25. A method of identifying a compound capable of treating a porcine Torque teno virus (TTV) infection, said method comprising:
(a) exposing a young TTV-negative piglet, a barrier-raised specific pathogen-free piglet, or caesarian-delivered piglet to a porcine TTV isolate in an amount sufficient to cause infection in said piglet;
(b) delivering a compound or series of compounds to said infected piglet; and
(c) examining the piglet from step (b) for the presence or loss of TTV and/or the development, inhibition, or amelioration of PCVAD symptoms relative to an untreated TTV-infected piglet.
